(19) United States
(12) Patent Application Publication
(10) Pub. No.: US 2019/0321616 A1
(43) Pub. Date: Oct. 24, 2019

(54) MEDICAL DEVICE AND METHOD FOR MANUFACTURING THE SAME

(71) Applicant: TERUMO KABUSHIKI KAISHA, Tokyo (JP)

(72) Inventors: Yuta Akahori, Kofu-shi (JP); Shigeru Yoshikawa, Fujinomiya-shi (JP)

(21) Appl. No.: 16/503,087

(22) Filed: Jul. 3, 2019

Related U.S. Application Data
(63) Continuation of application No. PCT/JP2018/010589, filed on Mar. 16, 2018.

(30) Foreign Application Priority Data
Mar. 16, 2017 (JP) 2017-051695

(51) Int. Cl. A61M 39/10

(52) U.S. Cl. CPC A61M 39/10; A61M 25/0097; A61M 2207/00; A61M 2039/1072

(57) ABSTRACT

An integrally-molded medical device is provided including a tubular member and a fixing member molded around and fixed to a first end of the tubular member. The fixing member is molded around the tubular member to include an outer circumferential contact portion in contact with an outer circumferential face of the tubular member, and to include an inner circumferential contact portion in contact with an inner circumferential face of the tubular member. The circumferential wall of the tubular member includes, at different positions in a circumferential direction, a pinching portion which is pinched by the outer circumferential contact portion and the inner circumferential contact portion, and a non-pinching portion which is in contact with the outer circumferential contact portion, is not in contact with the inner circumferential contact portion, and is not pinched by the outer circumferential contact portion and the inner circumferential contact portion.

### CROSS REFERENCE TO RELATED APPLICATIONS

The present application is a continuation of and claims benefit to PCT Application No. PCT/JP2018/010589, filed on Mar. 16, 2018, entitled “Medical Instrument and Method for Manufacturing Medical Instrument” which claims priority to Japanese Patent Application 2017-051695, filed Mar. 16, 2017. The entire disclosures of the applications listed above are hereby incorporated herein by reference, in their entirety, for all that they teach and for all purposes.

### TECHNICAL FIELD

The present disclosure relates to a medical device and, in particular, an infusion flow connector device and a method for manufacturing the same.

### BACKGROUND

In some cases, is necessary to form a route (e.g., an infusion line) that is configured to transport a fluid, such as a medicinal solution, when performing infusion on a patient. The infusion line is generally formed by connecting a medical tube and various medical devices together.

For example, Japanese Patent Application JP-2012-19829 A discloses a medical medicinal solution transfer device that includes a branch pipe, a connector, and a tube and is capable of transferring a medicinal solution between the branch tube and the connector by connecting one end of the tube to an opening of the branch tube and connecting the other end of the tube to the connector.

### SUMMARY

### Technical Problem

In liquid flow paths such as infusion lines formed by members such as tubes, connectors, and various medical devices, there is an issue where the internal pressure of the infusion lines rises when a high-viscosity fluid such as a contrast agent is supplied or when a fluid vigorously flows into the infusion lines. When the internal pressure rises, a load, or force, may be applied to a connection point between the members, and an unintended loosening of the members may occur while in the connected state. When such loosening of the members occurs, there is a risk that a liquid could leak from the connection point. In addition, there is also a risk that the connection between the members may completely release or otherwise become disconnected.

Embodiments of the present disclosure address this issue and provide a medical device (e.g., an infusion flow connector device) having a configuration in which the loosening of a connection or release of the connection is substantially prevented at a connection point between members. Among other things, the medical device, and the method for manufacturing the medical device, as described herein provides a stable flow path throughout the connection and members even when the internal pressure of the flow path rises.

### Solution to the Problem

Embodiments described herein include a medical device configured as an integrally-molded article, including: a tubular member; and a fixing member molded to the tubular member and fixed to a first end of the tubular member, in which the fixing member includes an outer circumferential contact portion in contact with an outer circumferential face of the tubular member, and an inner circumferential contact portion in contact with an inner circumferential face of the tubular member, and a circumferential wall of the tubular member includes, at different positions in a circumferential direction, a pinching portion which is pinched by, and between, the outer circumferential contact portion and the inner circumferential contact portion, and a non-pinching portion which is in contact with the outer circumferential contact portion, is not in contact with the inner circumferential contact portion, and is not pinched by the outer circumferential contact portion and the inner circumferential contact portion.

Aspects of the above medical device include wherein a thickness of the outer circumferential contact portion in a radial direction of the tubular member is thicker than a thickness of the inner circumferential contact portion in the radial direction at a position where the pinching portion is formed.

Aspects of the above medical device include wherein a minimum inner diameter of the outer circumferential contact portion is smaller than an outer diameter of the tubular member in a natural (e.g., uncompressed from contact with the fixing member, etc.) state.

Aspects of the above medical device include wherein the outer circumferential contact portion covers the outer circumferential face of the tubular member in a whole region in the circumferential direction.

Aspects of the above medical device include wherein the outer circumferential contact portion is longer toward a second end of the tubular member than the inner circumferential contact portion.

Aspects of the above medical device include wherein the fixing member includes: a body portion provided at a position overlapping the first end of the tube member in a central axis direction of the tubular member; and a head portion extending in the central axis direction from the body portion and provided at a position that is not overlapping the tubular member, and a gate portion of the fixing member used during integral molding is provided in the head portion.

Aspects of the above medical device include wherein a volume of the head portion is larger than a volume of the body portion.

A medical device according to an embodiment of the present disclosure comprises an integrally-molded article, including: a tubular member; and a fixing member fixed to a first end of the tubular member, in which the fixing member includes an outer circumferential contact portion in contact with an outer circumferential face of the tubular member, and an inner circumferential contact portion in contact with an inner circumferential face of the tubular member, a circumferential wall of the tubular member includes a pinching portion which is pinched by the outer circumferential contact portion and the inner circumferential contact portion, and a thickness of the outer circumferential contact portion in a radial direction of the tubular member is thicker than a thickness of the inner circumferential contact portion in the radial direction at a position where the pinching portion is formed.

A medical device according to an embodiment of the present disclosure comprises an integrally-molded article, including: a tubular member; and a fixing member fixed to a first end of the tubular member, in which the fixing member includes: a body portion provided at a position overlapping the first end of the tubular member in a central axis direction of the tubular member; and a head portion extending in the central axis direction from the body portion and provided at a position that is not overlapping the tubular member, and a gate portion of the fixing member used during integral molding is provided in the head portion.

A medical device according to an embodiment of the present disclosure comprises: a tubular member; and a fixing member fixed to the tubular member in a state where a first end of the tubular member is accommodated in the fixing member, in which the first end of the tubular member includes a protruding portion that protrudes radially outward, and the fixing member includes a movement restriction portion which is in contact with a face of the protruding portion on a second end of the tubular member, and restricts movement of the first end of the tubular member in a removal direction.

Embodiments include a method for manufacturing an integrally-molded medical device comprising a tubular member and a fixing member fixed to a first end of the tubular member, the method including: a loading step of loading the tubular member into a molding die including a cavity, or internal space, that forms an outer shape of the fixing member; and a filling step of filling the molding die with a molding material of the fixing member so as to be in contact with an inner circumferential face and an outer circumferential face of the tubular member and integrally molding the fixing member and the tubular member together, wherein, after the molding material of the fixing member cures, the inner circumferential face and the outer circumferential face of the tubular member are pinched between portions of the fixing member at the first end of the tubular member.

Aspects of the above method include wherein in the loading step, the tubular member is externally fitted to a core pin, and an inner flow path is formed between the inner circumferential face of the tubular member and the core pin, and an outer flow path is formed on a side of the outer circumferential face of the tubular member, and in the filling step, the inner flow path and the outer flow path are filled with the molding material.

### Non-Exhaustive Advantages

According to the present disclosure, embodiments of a medical device are provided that substantially prevent and/or otherwise resist loosening of the connection, or the release of the connection, at a connection point between various interconnected fluid flow line members. The embodiments of the medical device, and the method for manufacturing the medical device, provide a consistent flow path even when the internal pressure of the fluid flow lines and the flow path rises beyond an unacceptable pressure level (e.g., pressure levels that cause conventional connections to loosen and/or separate completely, etc.).

### BRIEF DESCRIPTION OF THE DRAWINGS

FIG. 1 is a plan view of an infusion set including a medical device according to an embodiment of the present disclosure.

FIG. 2 is a perspective view of a proximal end of the infusion set illustrated in FIG. 1.

FIG. 3 is a cross-sectional view of the proximal end of the infusion set illustrated in FIG. 1.

FIG. 4 is a perspective view of a holder isolated from the infusion set illustrated in FIG. 2.

FIG. 5 is a broken detail cross-sectional view illustrating an enlarged area of a proximal end of the joint tube shown in FIG. 3.

FIG. 6 is a flowchart illustrating a method for manufacturing the medical device according to embodiments of the present disclosure.

FIG. 7 is a schematic cross-sectional view illustrating the core pin arrangement step of FIG. 6.

FIG. 8 is a schematic cross-sectional view illustrating the loading step of FIG. 6.

FIG. 9 is a schematic cross-sectional view illustrating the filling step of FIG. 6.

FIG. 10 is a schematic cross-sectional view illustrating a state where the medical device is taken out of a molding die after the filling step illustrated in FIG. 9.

FIG. 11 is a schematic cross-sectional view taken along line I-I of FIG. 9.

FIG. 12 is a schematic cross-sectional view illustrating a position of an inlet for adding a molding material when the medical device is integrally molded according to embodiments of the present disclosure.

FIG. 13 is a cross-sectional view illustrating an embodiment of the medical device illustrated in FIG. 3.

### DETAILED DESCRIPTION

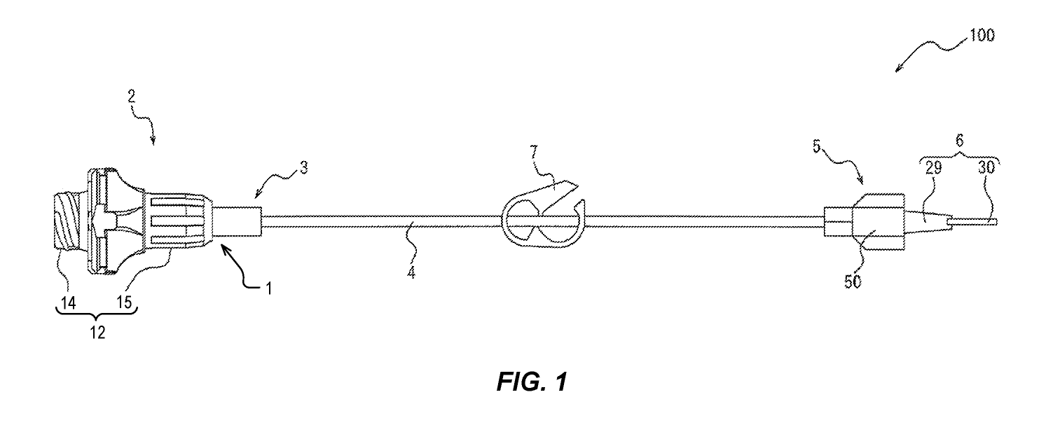

Hereinafter, embodiments of a medical device and a method for manufacturing the medical device according to the present disclosure will be described with reference to FIGS. 1 to 13. The same reference characters are attached to members and parts common in the respective drawings.

FIG. 1 is a view illustrating an infusion set 100 including a medical device 1 according to an embodiment of the present disclosure. The infusion set 100 illustrated in FIG. 1 forms an infusion line as a flow path of a liquid by connecting a plurality of members to one another. Specifically, the infusion set 100 includes a first medical connector 2, a joint tube 3, a medical tube 4, a second medical connector 5, an indwelling needle member 6, and a clamp 7. The medical device 1 includes a tubular member and a fixing member fixed to the tubular member. The tubular member of the medical device 1 of the present embodiment is the joint tube 3. In addition, the fixing member of the medical device 1 of the present embodiment is a holder 15, which will be described later, of the first medical connector 2.

In the infusion set 100 illustrated in FIG. 1, the first medical connector 2, the joint tube 3, the medical tube 4, the second medical connector 5, and the indwelling needle member 6 are arranged in this order from a proximal end side to a distal end side, and the adjacent members are connected to one another. As illustrated in FIG. 1, the formed infusion line extends from the proximal end to the distal end.

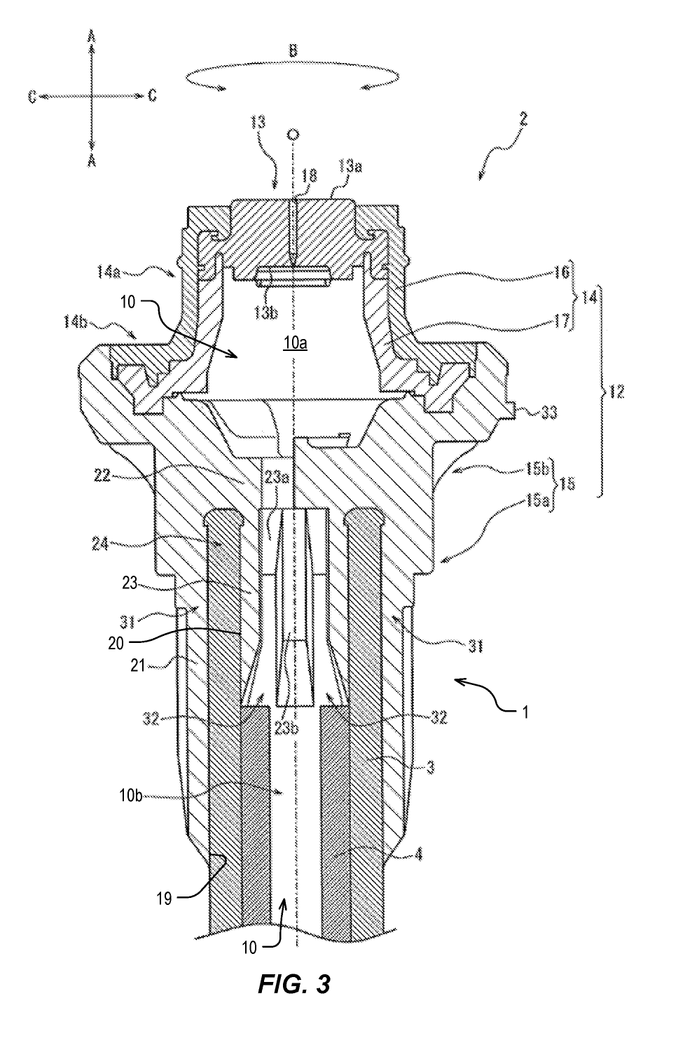

The respective members constituting the infusion set 100, as illustrated in FIG. 1, will now be described. Details of the medical device 1 may be described at least in conjunction with FIGS. 3 to 5, etc.

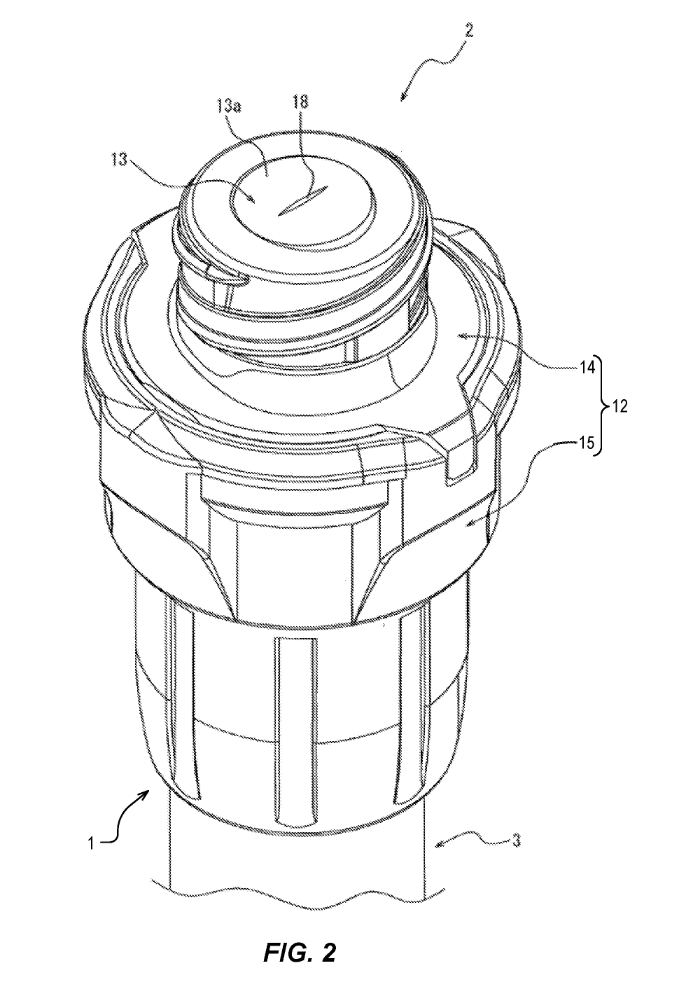

FIG. 2 is a perspective view of the proximal end of the infusion set 100 illustrated in FIG. 1. FIG. 3 is a cross-sectional view of the proximal end of the infusion set 100 illustrated in FIG. 1. More specifically, FIG. 3 is a cross-sectional view taken along a plane passing through central axis O including central axes O of the first medical connector 2 and the joint tube 3.

As illustrated in FIGS. 1 to 3, the first medical connector 2 constitutes an end on the proximal end side of the infusion set 100 forming the infusion line. The infusion set 100 may provide an interconnection at the end that allows another member (e.g., a mating fitting, a mating attachment, another line, etc.) to be connected to the proximal end side of the infusion line formed by the infusion set 100 using the first medical connector 2.

In some embodiments, the first medical connector 2 may correspond to a female connector to which a male connector is connectable from the outside. For instance, the first medical connector 2 may correspond to a locking female connector that conforms to one or more International Organization for Standardization (ISO) standards for small-bore connectors for liquids and gases in healthcare applications, for example, connectors for intravascular or hypodermic applications, as defined by ISO 80369-7:2016. Thus, the infusion line formed by the infusion set 100 illustrated in FIG. 1 can be further extended from the proximal end side (e.g., in a direction away from the distal end side) by, for example, connecting a male connector positioned at the distal end of an infusion line formed by an infusion set different from the infusion set 100, to the first medical connector 2. Examples of the male connector connectable to the first medical connector 2 may include, but are in no way limited to, a mating locking male connector conforming to ISO 80369-7, such as the second medical connector 5 of the infusion set 100.

In one embodiment, a hollow portion 10 penetrating along the central axis O is defined inside the first medical connector 2. The first medical connector 2 is fixed to the joint tube 3 in a state where the proximal end as one end of the joint tube 3 in a central axis direction A is accommodated therein, that is, in the hollow portion 10.

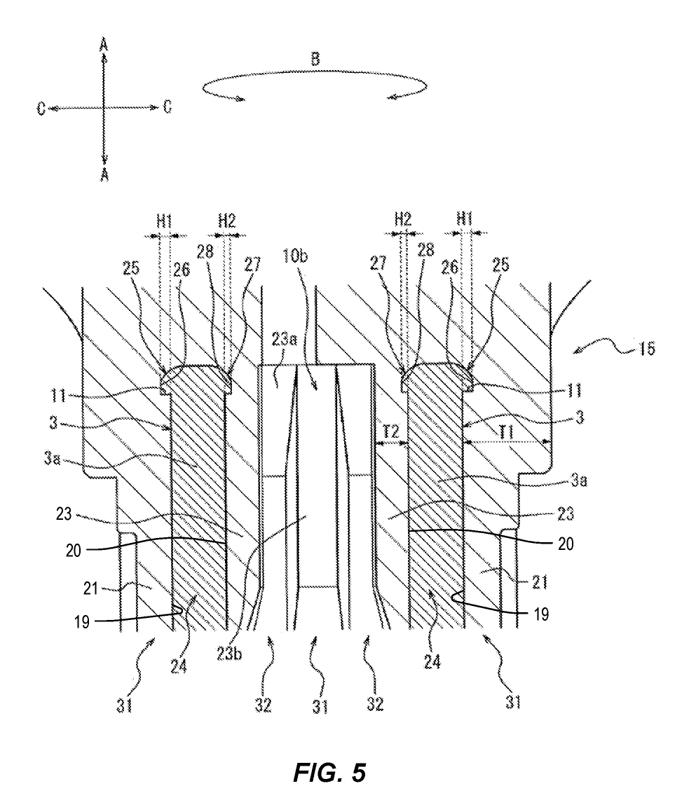

More specifically, a movement restriction portion (see, e.g., FIG. 5), which is engaged with a protruding portion 25 (see, e.g., FIG. 5) of the joint tube 3, and which restricts movement of the joint tube 3 in a removal direction (e.g., along the central axis direction A, etc.), is provided on an inner wall defining the hollow portion 10 of the first medical connector 2. Among other things, these portions 11, 25 may prevent, or mitigate, the first medical connector 2 from separating or disconnecting from the joint tube 3. The protruding portion 25 and the movement restriction portion 11 are described in greater detail in conjunction with FIG. 5.

Hereinafter, additional details of the first medical connector 2 in accordance with embodiments of the present disclosure will be described.

In some embodiments, the first medical connector 2 may include a housing 12 and an elastic valve body 13. The hollow portion 10 of the first medical connector 2 described above is defined by the housing 12. As illustrated in FIG. 2, the elastic valve body 13 may be arranged at a position on the proximal end side of the hollow portion 10 and closes the hollow portion 10.

The housing 12 may include a cap 14 and a holder 15 supporting the cap 14. The above-described hollow portion 10 may be defined by the cap 14 and the holder 15.

The cap 14 may include a tube portion 14a that defines a cap hollow portion 10a accommodating the elastic valve body 13 in the hollow portion 10, and a flange portion 14b provided at the distal end of the tube portion 14a and supported on the holder 15. In one embodiment, the cap 14 may comprise a top face cap 16 and a bottom face cap 17. Each of the top face cap 16 and the bottom face cap 17 has a hat shape having a tube portion and a flange portion, and the cap 14 is formed by superimposing, or stacking, the top face cap 16 and the bottom face cap 17 on one another and then by joining specific contact faces of the two caps 16, 17 by ultrasonic welding or the like. That is, the tube portion 14a of the cap 14 is constituted by the tube portions of the top face cap 16 and the bottom face cap 17 superimposed, or stacked, on one another. In addition, the flange portion 14b of the cap 14 is constituted by the flange portions of the top face cap 16 and the bottom face cap 17 superimposed, or stacked, on one another.

The elastic valve body 13 may be compressed and pinched between a portion of the top face cap 16 and the bottom face cap 17, and maintained in a position in the hollow portion 10. In some embodiments, the elastic valve body may be fixed in an area of the cap hollow portion 10a. A male connector can be inserted into the hollow portion 10 defined by the housing 12 from the outside through a slit 18 to be described later formed in the elastic valve body 13.

The holder 15 supports the cap 14. In addition, the holder 15 defines a flow path 10b communicating on the proximal end side with the cap hollow portion 10a defined by the cap 14 in the state of supporting the cap 14 (see, e.g., FIG. 3, etc.). The distal end side of the flow path 10b communicates with the outside, and the proximal end as one end of the joint tube 3 is accommodated in the distal end side of the flow path 10b. Further, the holder 15 is fixed with respect to the joint tube 3 in the state where the proximal end of the joint tube 3 is accommodated in the flow path 10b. Specifically, the holder 15 includes the movement restriction portion 11 (see, e.g., FIG. 5) which is in contact with the protruding portion 25 (see, e.g., FIG. 5) of the joint tube 3, which will be described later, positioned in the flow path 10b and restricts the movement of the joint tube 3 in the removal direction (the movement to the distal end side). Thus, the first medical connector 2 including the holder 15 is fixed to the joint tube 3 so as not to be withdrawn from the joint tube 3. Details of an engagement relationship between the protruding portion 25 of the joint tube 3 and the movement restriction portion 11 of the holder 15 will be described later (see, e.g., FIG. 5).

In addition, in the state where the proximal end of the joint tube 3 is accommodated in the flow path 10b (see, e.g., FIG. 3), the holder 15 includes an outer circumferential contact portion 19 in contact with an outer circumferential face of the joint tube 3 and an inner circumferential contact portion 20 in contact with an inner circumferential face of the joint tube 3. Further, the outer circumferential contact portion 19 and the inner circumferential contact portion 20 oppose each other in a radial direction (which is the same direction as a radial direction C of the joint tube 3 and will be described simply as the “radial direction C” hereinafter), and pinch a circumferential wall of the joint tube 3 in the radial direction C.

As illustrated in FIG. 3, the holder 15 includes: an annular tube portion 21 which covers the outer circumferential face of the proximal end of the joint tube 3 over the whole region in a circumferential direction B; an annular flange portion 22 which is provided to protrude from the tube portion 21 inward in the radial direction C and opposes the proximal end of the joint tube 3 in a central axis direction (which is the same direction as the central axis direction A of the joint tube 3 and will be described simply as the “central axis direction A” hereinafter); and a projection 23 that protrudes from the annular flange portion 22 to the distal end side and extends inside the joint tube 3 at an inner position in the radial direction C relative to the tube portion 21.

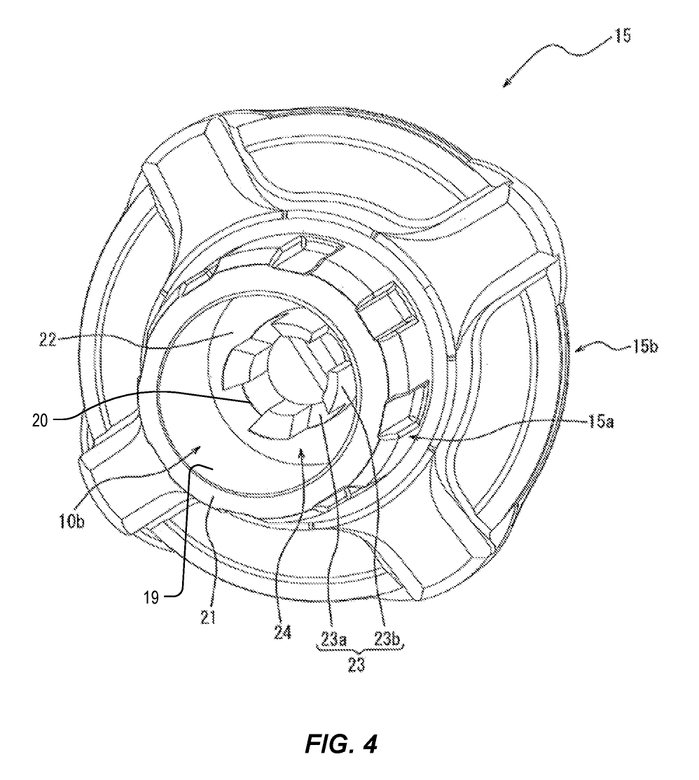

FIG. 4 is a perspective view of the holder 15 alone, for example, isolated from the infusion set 100, etc. As illustrated in FIGS. 3 and 4, the projection 23 includes: a tubular proximal end 23a provided to protrude from the annular flange portion 22; and a protruding distal end 23b further extending from the tubular proximal end 23a to the distal end side at a position of a part in a circumferential direction of the tube portion 21 of the holder 15 (which is the same direction as the circumferential direction B of the joint tube 3 and will be described simply as the “circumferential direction B” hereinafter). More specifically, a plurality of the protruding distal ends 23b of the projection 23 are provided at different positions in the circumferential direction B. In one embodiment, the four protruding distal ends 23b may be arranged at equal intervals in the circumferential direction B.

In other words, the proximal end of the joint tube 3 may be accommodated in an annular groove portion 24 defined by the tube portion 21, the annular flange portion 22, and the projection 23. An inner circumferential face of the tube portion 21 is in contact with the outer circumferential face of the joint tube 3 in a state where the proximal end of the joint tube 3 is accommodated in the annular groove portion 24 (see, e.g., FIG. 3). In addition, an outer surface of the projection 23 is in contact with the inner circumferential face of the joint tube 3 in the state where the proximal end of the joint tube 3 is accommodated in the annular groove portion 24 (see, e.g., FIG. 3). Further, the proximal end of the joint tube 3 is pinched between the tube portion 21 of the holder 15 and the projection 23 in the radial direction C. That is, the above-described outer circumferential contact portion 19 may be part of the tube portion 21 of the holder 15. In addition, the above-described inner circumferential contact portion 20 of the present embodiment may be part of the projection 23 of the holder 15. In this manner, the joint tube 3 may be pinched between the tube portion 21 and the projection 23 in the state of being accommodated in the annular groove portion 24 of the flow path 10b of the holder 15 (see, e.g., FIG. 3), and thus, may be prevented from being detached, or otherwise separated, from the holder 15, and the connection state between the joint tube 3 and the holder 15 is easily maintained.

In one embodiment, the above-described movement restriction portion 11 (see, e.g., FIG. 5) is configured by an outward annular concave portion serving as the recessed portion 26 (see, e.g., FIG. 5) formed on an inner wall of the tube portion 21. Details thereof will be described later (see, e.g., FIG. 5).

The outer circumferential contact portion 19 is configured to be longer toward the distal end side of the joint tube 3 (corresponding to the other end side when the proximal end side of the joint tube 3 is defined as one end side) than the inner circumferential contact portion 20. Specifically, the tube portion 21 serving as the outer circumferential contact portion 19 extends farther to the distal end side than the projection 23 serving as the inner circumferential contact portion 20 in the present embodiment. In other words, a distal end of the projection 23 terminates in the tube portion 21 and does not protrude beyond the distal end of the tube portion 21.

In some embodiments, both the top face cap 16 and the bottom face cap 17 are configured to be supported by the holder 15 in a contact manner, but the configuration in which the bottom face cap 17 is held by the top face cap 16, and only the top face cap 16 is brought into contact with the holder 15 so as to be supported by the holder 15 may be adopted. On the contrary, the configuration in which the top face cap 16 is held by the bottom face cap 17, and only the bottom face cap 17 is brought into contact with the holder 15 so as to be supported by the holder 15 may be adopted.

Examples of materials for the holder 15 of the housing 12, the top face cap 16, and the bottom face cap 17 include various resin materials, for example, a polyolefin such as polyethylene, polypropylene, and an ethylene-propylene copolymer; an ethylene-vinyl acetate copolymer (EVA); polyvinyl chloride; polyvinylidene chloride; polystyrene; polyamide; polyimide; polyamide-imide; polycarbonate; poly(4-methylpentene-1); ionomer; an acrylic resin; polymethyl methacrylate; an acrylonitrile-butadiene-styrene copolymer (ABS resin); an acrylonitrile-styrene copolymer (AS resin); a butadiene-styrene copolymer; polyester such as polyethylene terephthalate (PET), polybutylene terephthalate (PBT), and polycyclohexane terephthalate (PCT); polyether; polyether ketone (PEK); polyether ether ketone (PEEK); polyether imide; polyacetal (POM); polyphenylene oxide; modified polyphenylene oxide; polysulfone; polyether sulfone; polyphenylene sulfide; polyarylate; aromatic polyester (a liquid crystal polymer); and polytetrafluoroethylene, polyvinylidene fluoride and other fluororesins. Additionally or alternatively, a blend or a polymer composite containing one or more kinds of the above materials may also be used as a material for the holder 15. In some embodiments, various glass materials, ceramic materials, or other metal materials may be used as the material for the holder 15.

Although the housing 12 of the present embodiment is configured to include the holder 15, the top face cap 16, and the bottom face cap 17, the present disclosure is not limited to this configuration, and the holder 15 and the bottom face cap 17 of the present embodiment can be formed as a single member molded using a single material, for example. In addition, it is also possible to form any of the holder 15, the top face cap 16, and the bottom face cap 17 of the present embodiment using a combination of a plurality of members. In this manner, the housing 12 is not limited to the configuration illustrated in FIG. 3, and for example, can be configured as one member or two members or configured as four members, or more.

As illustrated in FIGS. 2 and 3, the elastic valve body 13 is a circular flat disc-shaped valve body having a substantially circular outer shape when viewed from a top face 13a side. The elastic valve body 13 is held as a portion of the elastic valve body 13 between the top face 13a and the bottom face 13b are pinched by the housing 12, whereby a position of the elastic valve body 13 is fixedly disposed at least partially in the hollow portion 10.

In addition, the elastic valve body 13 may include a slit 18 disposed at a central portion thereof when viewed from the top face 13a side. The slit 18 may be opened and closed by elastic deformation of the elastic valve body 13 when the male connector is inserted into and/or removed from the hollow portion 10. The elastic valve body 13 is held as the top face 13a and the bottom face 13b are pinched by the housing 12 at a position of a circumferential edge positioned radially outward of the central portion where the slit 18 is formed.

The elastic valve body 13 is molded and formed to be elastically deformable. Examples of the material of the elastic valve body 13 may include, but are in no way limited to, various rubber materials such as natural rubber, isoprene rubber, butadiene rubber, styrene-butadiene rubber, nitrile rubber, chloroprene rubber, butyl rubber, acrylic rubber, ethylene-propylene rubber, hydrin rubber, urethane rubber, silicone rubber, and fluoro rubber; and various thermoplastic elastomers such as a styrene-based thermoplastic elastomer, a polyolefin-based thermoplastic elastomer, a polyvinyl chloride-based thermoplastic elastomer, a polyurethane-based thermoplastic elastomer, a polyester-based thermoplastic elastomer, a polyamide-based thermoplastic elastomer, a polybutadiene-based thermoplastic elastomer, a transpolyisoprene-based thermoplastic elastomer, a fluoro rubber-based thermoplastic elastomer, and a chlorinated polyethylene-based thermoplastic elastomer, and/or a material mixed with one or two or more kinds of these materials, which may be used to form the elastic valve body 13.

In addition, it is preferable to set the hardness of the elastic valve body 13 to the hardness that enables the elastic valve body 13 to secure an appropriate elastic force. The hardness of the elastic valve body 13 may be set to the hardness that enables elastic deformation so as to open the slit 18 when the male connector is inserted into the hollow portion 10. In addition, the hardness may be set so as to be capable of realizing a liquid-tight connection state as the elastic valve body 13 is brought into close contact with an outer wall of the male connector in the state where the male connector is inserted into the hollow portion 10 through the slit 18. Further, the hardness may be set such that the elastic valve body 13 can be restored within the housing 12 such that the slit 18 is closed when the male connector is removed from the hollow portion 10. Although the hardness of the elastic valve body 13 is not particularly limited, as long as the hardness retains such performance, the hardness may be set to a Shore durometer hardness of 20 A to 60 A hardness, in accordance with embodiments of the present disclosure.

The joint tube 3 connects the first medical connector 2 positioned on the proximal end side and the medical tube 4 positioned on the distal end side such that flow paths inside both the members communicate with each other in a liquid-tight manner. Specifically, a state where the proximal end as one end of the joint tube 3 is fitted in the annular groove portion 24 in the flow path 10b of the holder 15 of the first medical connector 2 is formed as illustrated in FIG. 3. In addition, a state where a proximal end as one end of the medical tube 4 is fitted inside the distal end as the other end of the joint tube 3 is formed as illustrated in FIG. 3. In this manner, the joint tube 3 of the present embodiment is set to the state of being fitted with the first medical connector 2 and the medical tube 4, thereby connecting the first medical connector 2 and the medical tube 4.

FIG. 5 is an enlarged broken detail cross-sectional view illustrating a vicinity of the proximal end of the joint tube 3 in FIG. 3 in an enlarged manner. As illustrated in FIG. 5, the proximal end as one end of the joint tube 3 has the protruding portion 25 that protrudes outwardly in the radial direction C. More specifically, an outward annular convex portion serving as the protruding portion 25, which protrudes outwardly in the radial direction C from an outer circumferential face of a body portion 3a having a substantially uniform outer diameter over the inside and the outside of the holder 15, is formed at the proximal end of the joint tube 3 of the present embodiment.

In addition, the outward annular concave portion serving as the recessed portion 26, which is concave outwardly in the radial direction C, to accommodate the outward annular convex portion serving as the protruding portion 25 is formed on an inner surface of the tube portion 21 of the holder 15 as illustrated in FIG. 5. That is, the recessed portion 26 corresponding to the protruding portion 25 of the joint tube 3 is provided in the tube portion 21 of the holder 15. Thus, even if the joint tube 3 is to be removed from the flow path 10b of the holder 15 to the distal end side, a face of the outward annular convex portion, which serves as the protruding portion 25 of the joint tube 3, on the distal end side of the joint tube 3 (the other end side of the joint tube 3 when the proximal end side of the joint tube 3 is set as the one end side, and the lower side in FIG. 5) abuts on an inner wall on the distal end side (the lower side in FIG. 5) of the joint tube 3 in the inner wall defining the outward annular concave portion serving as the recessed portion 26 of the tube portion 21. That is, the protruding portion 25 abuts on the inner wall of the outward annular concave portion serving as the movement restriction portion 11, and thus, the joint tube 3 may be prevented from being detached from the holder 15.

Further, the proximal end as one end of the joint tube 3 of the present embodiment has a protruding portion 27, which protrudes inwardly in the radial direction C as illustrated in FIG. 5. In one embodiment, an inward annular convex portion serving as the protruding portion 27, which protrudes inwardly in the radial direction C from an inner circumferential face of the body portion 3a having a substantially uniform inner diameter over the inside and the outside of the holder 15, is formed at the proximal end of the joint tube 3.

As illustrated in FIG. 5, an inward annular concave portion serving as a recessed portion 28, which is concave inward in the radial direction C, to accommodate the inward annular convex portion serving as the protruding portion 27 is formed on the tubular proximal end 23a of the projection 23 of the holder 15. That is, the recessed portion 28 corresponding to the protruding portion 27 of the joint tube 3 is provided in the tubular proximal end 23a of the projection 23 of the holder 15. Thus, even if the joint tube 3 is to be removed from the flow path 10b of the holder 15 to the distal end side, a face of the inward annular convex portion, which serves as the protruding portion 27 of the joint tube 3, on the distal end side of the joint tube 3 (the other end side of the joint tube 3 when the proximal end side of the joint tube 3 is set as the one end side, and the lower side in FIG. 5) abuts on an inner wall on the distal end side (the lower side in FIG. 5) in an inner wall defining the inward annular concave portion serving as the recessed portion 28 of the projection 23. In this manner, the protruding portion 27 and the recessed portion 28 are provided inward in the radial direction C of the joint tube 3 in addition to the above-described protruding portion 25 and recessed portion 26 provided outward in the radial direction C of the joint tube 3, and thus, the joint tube 3 can be configured to be retained in the holder 15 and substantially prevented from being inadvertently detached from the holder 15.

Examples of a material for the joint tube 3 may include, but are in no way limited to, a soft polyvinyl chloride, an ethylene-vinyl acetate copolymer, polyethylene, polypropylene, polybutadiene, and the like, and/or materials containing one or more of these materials.

The proximal end as one end of the medical tube 4 is accommodated inside the distal end side of the joint tube 3 as described above. In addition, a portion, accommodated inside the joint tube 3, of an outer circumferential face of the medical tube 4 is joined to an inner circumferential face of the joint tube 3 by adhesion using, for example, an ultraviolet curing adhesive or the like or welding.

As illustrated in FIG. 3, the medical tube 4 is accommodated inside the joint tube 3 and inside the tube portion 21 of the holder 15 of the first medical connector 2. More specifically, the proximal end of the medical tube 4 is inserted into the tube portion 21 up to a position where the proximal end abuts on a distal end of the projection 23 of the holder 15, and is joined to the joint tube 3 in such a state. In this manner, it is preferable to provide at least a part of a joint between the joint tube 3 and the medical tube 4 inside the tube portion 21 of the holder 15. In this manner, deformation of portions positioned in the tube portion 21 of the holder 15, of the joint tube 3 and the medical tube 4 is restricted by the tube portion 21. Thus, a portion, positioned inside the tube portion 21, of the joint between the joint tube 3 and the medical tube 4 may be substantially prevented from being peeled off by an external force as compared to a portion that is not covered by the tube portion 21.

In addition, the second medical connector 5 is connected to a distal end as the other end of the medical tube 4.

Examples of a material for the medical tube 4 may include, without limitation, the same or similar materials as those of the joint tube 3 described above.

As illustrated in FIG. 1, the second medical connector 5 is the lock male connector conforming to ISO 80369-7, and includes a male Luer portion and a tubular portion 50 which is positioned around the male Luer portion and has a female screw portion formed on an inner surface thereof.

As illustrated in FIG. 1, the indwelling needle member 6 may include a hub member 29 into which the male Luer portion of the second medical connector 5 is inserted, and an indwelling needle 30 attached to a distal end of the hub member 29.

As illustrated in FIG. 1, a clamp 7 may be mounted on an outer surface of the medical tube 4 configured to be able to close the flow path inside the medical tube 4 by, for example, pinching the medical tube 4.

Embodiments of the medical device 1 will now be described. As described above, the infusion set 100 illustrated in FIG. 1 includes the medical device 1 according to the present embodiment. The medical device 1 includes the tubular member and the fixing member as described above. Specifically, the medical device 1 of the present embodiment is constituted by the joint tube 3 serving as the tubular member and the holder 15 serving as the fixing member.

As described above, the holder 15 serving as the fixing member is fixed to the joint tube 3 in a state where the proximal end of the joint tube 3 as one end of the tubular member is accommodated therein. Specifically, the proximal end of the joint tube 3 is shown as having the outward annular convex portion serving as the protruding portion 25 that protrudes outwardly in the radial direction C, as illustrated in FIG. 5. In addition, the holder 15 includes the movement restriction portion 11 which is in contact with the face of the outward annular convex portion serving as the protruding portion 25 on the distal end side of the joint tube 3, which is the face on the other end side of the tubular member, and restricts the movement of the proximal end of the joint tube 3 in the removal direction (e.g., a movement toward the distal end along the central axis direction A). The movement restriction portion 11 of may be configured as an inner wall, or flange, disposed on the distal end side of the outward annular concave portion serving as the recessed portion 26.

In this manner, the fixing member can be substantially prevented from being pulled out of the tubular member, or vice versa, by the cooperation of the movement restriction portion 11 of the fixing member and the protruding portion 25 of the tubular member in the medical device 1 in accordance with embodiments of the present disclosure.

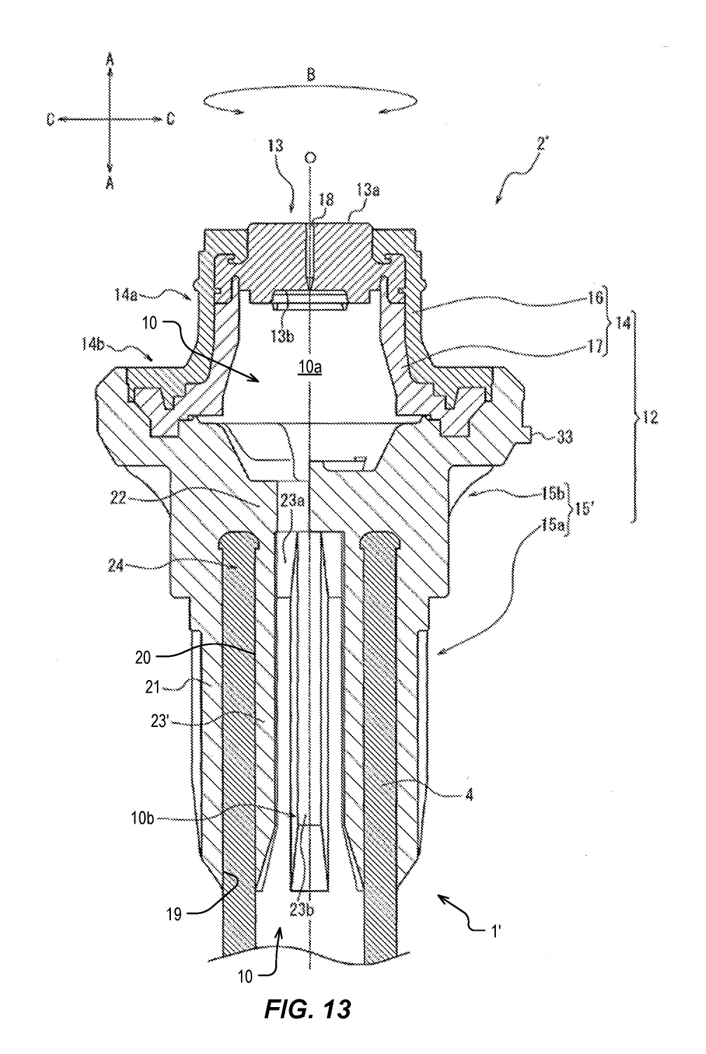

Although the joint tube 3 is illustrated as the tubular member and the holder 15 of the first medical connector 2 is illustrated as the fixing member, embodiments of the present disclosure are not limited to the medical device 1 illustrated. For instance, it is an aspect of the present disclosure that a medical device may be configured to include a tubular member having the protruding portion 25 and a fixing member having the movement restriction portion 11, as described herein, and does not cause loosening or detachment of connection even with a predetermined internal pressure (for example, approximately 1.5 MPa). Therefore, the medical tube 4 may be configured as a tubular member to which the holder 15 serving as a fixing member is fixed, for example, instead of the joint tube 3 illustrated in FIGS. 1 to 3 (see, e.g., FIG. 13). The holder 15, configured as a so-called I port, defining the flow path 10b and extending in a substantially linear shape, is illustrated as the fixing member in at least one embodiment of the present disclosure, but a holder, which may be configured as a so-called T port, having an upstream port and a downstream port may be used as the fixing member of the medical device. In some embodiments, another member irrelative to the medical connector may be used as the fixing member.

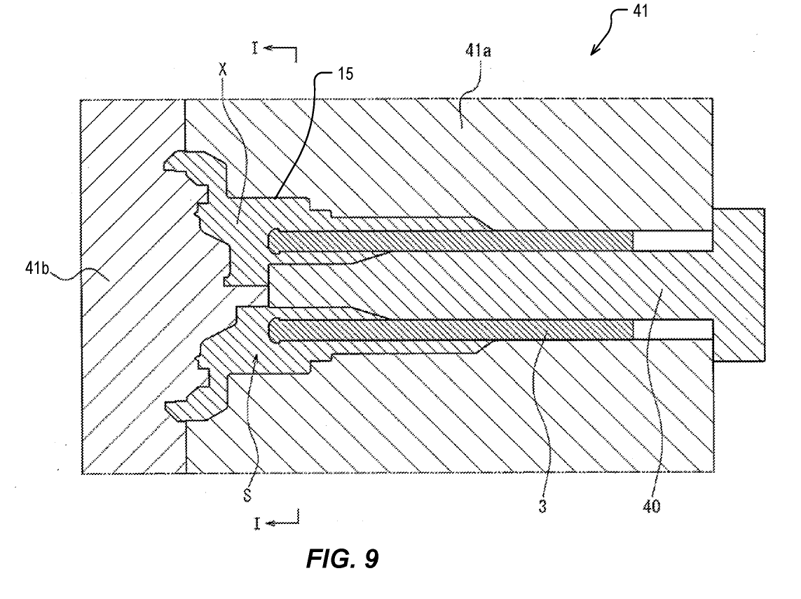

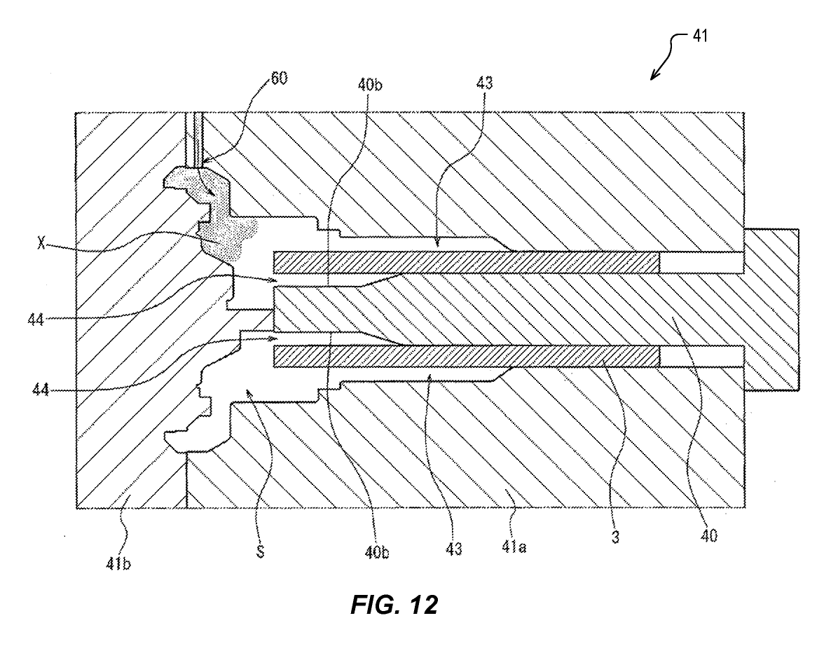

As the protruding portion 25, a convex portion, which protrudes outwardly in the radial direction C at the position of one end of the tubular member, like the outward annular protruding portion illustrated in FIG. 5, is formed. In addition, like the inner wall of the recessed portion 26 positioned on the distal end side of the tubular member (the other end side of the tubular member when the proximal end side of the tubular member is set as one end side, and the lower side in FIG. 5), the movement restriction portion 11 may be positioned on the distal end side of the tubular member with respect to the protruding portion 25, opposes the protruding portion 25 in the central axis direction A, and comes into contact with the face on the distal end side (the lower side in FIG. 5) of the protruding portion 25, such that the movement restriction portion 11 is configured to restrict the movement of the tubular member toward the distal end side. With such a configuration, the above-described protruding portion 25 and movement restriction portion 11 can be realized by integrally molding the tubular member and the fixing member even if each of the tubular member and the fixing member is formed of a single material such as a thermoplastic resin. Details of a manufacturing method for forming the protruding portion 25 of the tubular member and the movement restriction portion 11 of the fixing member illustrated in accordance with embodiments of the present disclosure by, for example, integral molding will be described later (see, e.g., FIGS. 9, 12, and the like).

As described above, the holder 15 serving as the fixing member includes the tube portion 21 serving as the outer circumferential contact portion 19 in contact with the outer circumferential face of the joint tube 3 serving as the tubular member (see, e.g., FIG. 3 and the like). In addition, the holder 15 serving as the fixing member includes the projection 23 serving as the inner circumferential contact portion 20 in contact with the inner circumferential face of the joint tube 3 serving as the tubular member (see, e.g., FIG. 3 and the like). Further, in the radial direction C, the circumferential wall of the joint tube 3 serving as the tubular member is pinched by the tube portion 21 serving as the outer circumferential contact portion 19 and the projection 23 serving as the inner circumferential contact portion 20 of the holder 15 serving as the fixing member.

In this manner, the circumferential wall of the tubular member (e.g., joint tube 3, etc.) is configured to be pinched by the outer circumferential contact portion 19 and the inner circumferential contact portion 20 of the fixing member, and thus, the pinched connection between the tubular member and the fixing member can substantially prevent loosening and/or releasing as compared with a configuration in which a circumferential wall of a tubular member is not pinched by an outer circumferential contact portion 19 and an inner circumferential contact portion 20.

In particular, when a tubular member and a fixing member are integrally molded by using the tubular member as an insert member, a circumferential wall of the tubular member is pinched by a molding material poured into a position where the outer circumferential contact portion 19 is formed and a molding material poured into a position where the inner circumferential contact portion 20 is formed when maintaining the pressure in the mold. Maintaining the pressure during the molding process may cause the various surfaces of the fixing member being formed to be forced into contact with the receiving surfaces of the tubular member. When the molding material cures, the surfaces of the tubular member may be compressed into intimate contact with the outer circumferential contact portion 19 as well as the inner circumferential contact portion 20 of the fixing member. Thus, it is possible to enhance the strength of pinching, or otherwise maintaining, the tubular member by the fixing member and to cause the connection between the tubular member and the fixing member to be more consistently held and substantially prevented from being loosened and/or released as compared with the case where the circumferential wall of the tubular member is pinched by the outer circumferential contact portion 19 and the inner circumferential contact portion 20 of the fixing member without using integral molding. For example, inserting a previously molded tubular member (e.g., joint tube 3) into an existing fixing member (e.g., holder 15) may result in at least one surface not contacting, or being pinched by, a portion of the fixing member (e.g., the outer circumferential contact portion 19 and/or the inner circumferential contact portion 20 of the fixing member). This occurrence may be a result of the tolerancing (e.g., the sizing of the respective components, etc.) required to insert the tubular member into an existing (e.g., previously molded, etc.) fixing member (e.g., holder 15). For instance, unless the tubular member is at least somewhat undersized (e.g., in outside diameter or inside diameter, etc.) relative to an existing fixing member, the tubular member could not be inserted into an existing fixing member without jamming, or creating a pressure build-up during insertion, that would prevent the tubular member from being fully inserted therein. Stated another way, without employing an integrally-molded medical device 1 (e.g., holder 15 and tubular member, etc.) as described herein, the tolerances associated with separate previously manufactured pieces that are later joined together would not provide the intimate contact with both the outer circumferential contact portion 19 and the inner circumferential contact portion 20 of the fixing member, which is disclosed as at least one benefit of integrally molding the fixing member to the tubular member as described herein.

As illustrated in FIGS. 3 and 5, the circumferential wall of the joint tube 3 serving as the tubular member includes, at different positions in the circumferential direction B at a predetermined position in the central axis direction A, a pinching portion 31 which is pinched by the outer circumferential contact portion 19 and the inner circumferential contact portion 20 in the radial direction C, and a non-pinching portion 32 which is in contact with the outer circumferential contact portion 19 and not in contact with the inner circumferential contact portion 20, and not pinched by the outer circumferential contact portion 19 and the inner circumferential contact portion 20 in the radial direction C. That is, the circumferential wall of the tubular member has both the pinching portion 31 and the non-pinching portion 32 in the circumferential direction B at the predetermined position in the central axis direction A. The tubular member may comprise a number, or series, of radially spaced apart pinching portions (e.g., areas where the tubular member is pinched by portions of the fixing member, etc.) and a series of non-pinching portions (e.g., areas where the tubular member is not pinched by portions of the fixing member, etc.). In some embodiments, the “predetermined position in the central axis direction A” is an arbitrary position in a range in which the protruding distal end 23b (see, e.g., FIGS. 3 to 5) is positioned in the central axis direction A.

FIG. 3 is a cross-sectional view taken along a plane passing through the central axis O that passes through the protruding distal end 23b. That is, the pinching portions 31 to be pinched between the tube portion 21 and the protruding distal end 23b are drawn in a cross-section illustrated in FIG. 3. In some embodiments, the pinching portions 31 are arranged at an interval in the circumferential direction B, and the non-pinching portion 32 is positioned at a position between the two pinching portions 31 adjacent in the circumferential direction B. In this manner, both the pinching portion 31 and the non-pinching portion 32 are formed in the circumferential direction B in the range in which the protruding distal end 23b (see, e.g., FIGS. 3 to 5) is positioned in the central axis direction A.

With such a configuration, it is possible to enhance the adhesion between the tubular member and the fixing member in the case of integrally molding the tubular member and the fixing member by using the tubular member as the insert member, and the connection between the tubular member and the fixing member after solidification of the molding materials can be configured so as to be substantially prevented from being loosened and/or released. In some embodiments, a portion of the molding material making up the tubular member may extend at least partially into the non-pinching portion 32 of the fixing member during the molding process, or vice versa. This portion of material and the other material making up the tubular member may key the tubular member to the features of the protruding distal end 23b of the fixing member. In one embodiment, the keyed tubular member (e.g., and portion of material extending into the non-pinching portion 32, etc.) may resist rotation of the tubular member in the circumferential direction B relative to the fixed member.

In some embodiments, at the time of integral molding, the circumferential wall of the tubular member is pinched by the molding material poured into the position where the outer circumferential contact portion 19 is formed and the molding material poured into the position where the inner circumferential contact portion 20 is formed when maintaining the pressure as described above. Thus, it is possible to enhance the strength of pinching the tubular member by the fixing member and to further strengthen the connection between the tubular member and the fixing member at the pinching portion 31 as compared with the case where the circumferential wall of the tubular member is pinched by the outer circumferential contact portion 19 and the inner circumferential contact portion 20 of the fixing member without using integral molding.

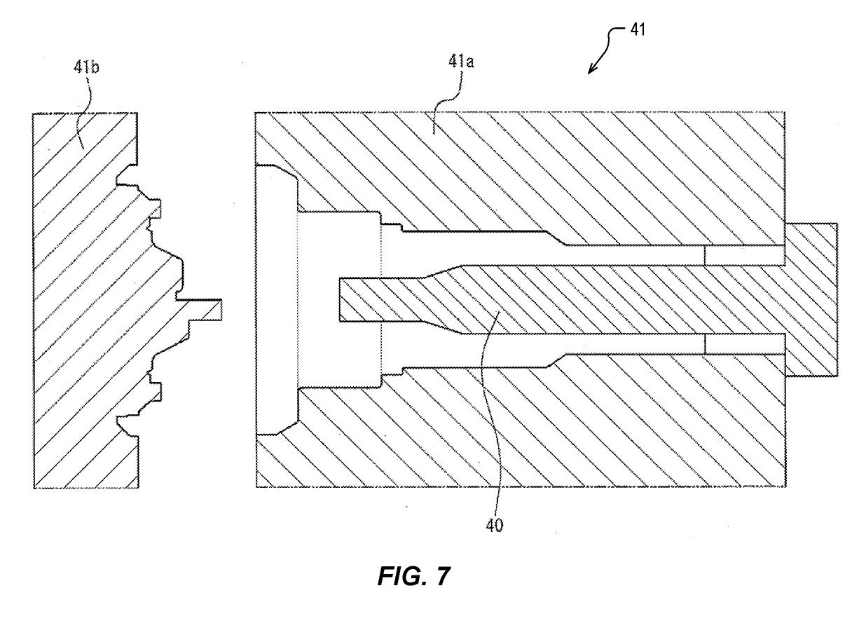

Further, a portion of the non-pinching portion 32 which is in contact with the outer circumferential contact portion 19 and not in contact with the inner circumferential contact portion 20 is compressed by the pinching pressure between the molding material poured into the position where the outer circumferential contact portion 19 is formed and a die (see, e.g., the “core pin 40” illustrated at least in FIGS. 7 to 9, and 11), which holds a cross-sectional shape of the tubular member in contact with a part of the inner circumferential face of the tubular member inside the tubular member, at the time of integral molding. That is, the adhesion between the outer circumferential contact portion 19 and the outer surface of the tubular member is also enhanced in the non-pinching portion 32 (e.g., via a keying of the material making up the tubular member with the fixing member, etc.).

In this manner, the circumferential wall of the tubular member is configured to have both the pinching portion 31 and the non-pinching portion 32 in the circumferential direction B, and thus, the connection between the tubular member and the fixing member in the case of being molded by integral molding can be further strengthened.

When the outer circumferential contact portion 19 and the inner circumferential contact portion 20 of the fixing member are integrally molded with the tubular member by using the tubular member as the insert member, it is preferable that a thickness of the outer circumferential contact portion 19 in the radial direction C be configured to be thicker than a thickness of the inner circumferential contact portion 20 in the radial direction C at a position where the pinching portion 31 is formed. In this manner, it is possible to further enhance the adhesion between the outer circumferential contact portion 19 and the outer surface of the tubular member even at the position of the pinching portion 31.

In some embodiments, a thickness T1 (see, e.g., FIG. 5) of the outer circumferential contact portion 19 in the radial direction C is set to be thicker than a thickness T2 (see, e.g., FIG. 5) of the inner circumferential contact portion 20 in the radial direction C at the position where the pinching portion 31 is formed, the internal pressure applied inwardly (e.g., in a direction toward the central axis O, etc.) from the outside in the radial direction C also increases at the position where the pinching portion 31 is formed due to the maintaining pressure at the time of integral molding. That is, the circumferential wall of the tubular member is easily compressed inwardly in the radial direction C by the molding material forming the outer circumferential contact portion 19 even in a portion, which is to serve as the pinching portion 31, of the circumferential wall of the tubular member. Thus, the circumferential wall of the tubular member can be compressed inward in the radial direction C not only at the non-pinching portion 32 but also at the position of the pinching portion 31, and the connection between the integrally-molded tubular member and fixing member can be further strengthened.

As illustrated in FIG. 5, the “thickness of the outer circumferential contact portion in the radial direction” and the “thickness of the inner circumferential contact portion in the radial direction” to be compared are thicknesses at positions opposing each other in the radial direction C with the tubular member therebetween.

In addition, when the tubular member and the fixing member are integrally molded by using the tubular member as the insert member as described above, the minimum inner diameter of the outer circumferential contact portion 19 is smaller than the outer diameter of the tubular member in the natural state in the integrally-molded medical device 1. That is, the circumferential wall of the tubular member is set to the state of being compressed inwardly in the radial direction C by the outer circumferential contact portion 19 in both the pinching portion 31 and the non-pinching portion 32. Thus, at least the minimum inner diameter of the outer circumferential contact portion 19 is smaller than the outer diameter of the tubular member in the natural state in the integrally-molded medical device 1. The above-described “minimum inner diameter of the outer circumferential contact portion 19” means a radius of a locus drawn in the case of rotating a point of the outer circumferential contact portion 19 positioned on the innermost side in the radial direction C about the central axis O. In addition, the above-described “outer diameter of the tubular member in the natural state” means an outer diameter of a portion, in contact with the outer circumferential contact portion 19 at the time of integral molding, in the single tubular member in the natural state before being subjected to integral molding and/or being compressed by the fixing member, etc. Meanwhile, in the case of a configuration in which the single tubular member in the natural state before being subjected to integral molding has a substantially uniform outer diameter regardless of the position in the central axis direction, the maximum outer diameter in a portion not in contact with the outer circumferential contact portion 19 and the inner circumferential contact portion 20, the portion where there is no other member in contact with the outer circumferential face and the inner circumferential face, in the integrally-molded tubular member can be approximated to the above-described “outer diameter of the tubular member in the natural state.”

In some embodiments, the tube portion 21 serving as the outer circumferential contact portion 19 covers the periphery of the proximal end of the joint tube 3 over the whole region in the circumferential direction B at an arbitrary position in the range in which the protruding distal end 23b is positioned in the central axis direction A, and is in contact with the outer circumferential face of the proximal end of the joint tube 3 over the whole region in the circumferential direction B. Thus, the molding material forming the outer circumferential contact portion 19 compresses the outer surface of the tubular member inwardly in the radial direction C (e.g., toward the central axis O, etc.) over the entire region in the circumferential direction B when maintaining the pressure in integral molding. As a result, it is possible to prevent the adhesion between the outer circumferential face of the tubular member and the outer circumferential contact portion 19 from being locally reduced in a part in the circumferential direction B as compared with a configuration in which the outer circumferential contact portion 19 does not cover the tubular member over the entire region in the circumferential direction B of the tubular member. Therefore, the connection between the tubular member and the fixing member can be further strengthened.

In one embodiment, the pinching portion 31 is at the position where the protruding distal end 23b of the projection 23 is arranged in the range in which the protruding distal end 23b is positioned in the central axis direction A. In addition, the non-pinching portion 32 of the present embodiment is at the position where the protruding distal end 23b of the projection 23 is not arranged in the range in which the protruding distal end 23b is positioned in the central axis direction A.

As described above, the medical device 1 includes the joint tube 3 serving as the tubular member and the holder 15 serving as the fixing member. Further, the medical device 1 of the present embodiment is an integrally-molded article in which the joint tube 3 and the holder 15 are integrally molded with one another.

As illustrated in FIGS. 3 and 4, the holder 15 serving as the fixing member includes: a body portion 15a provided at a position overlapping with the proximal end of the joint tube 3 as one end of the tubular member in the central axis direction A of the joint tube 3 serving as the tubular member; and a head portion 15b which extends from the body portion 15a in the central axis direction A and is provided at a position not overlapping with the joint tube 3 serving as the tubular member. The body portion 15a of the present embodiment is configured by the tube portion 21 and the projection 23 of the holder 15. In addition, the head portion 15b of the present embodiment is configured by a portion of the holder 15 that includes the annular flange portion 22 and is positioned closer to the proximal end side than the tube portion 21 and the projection 23.

Further, the gate portion 33 of the fixing member used in integral molding is provided in the head portion 15b as illustrated in FIG. 3. Specifically, the holder 15 serving as the fixing member of the present embodiment is integrally molded with the joint tube 3 by using the joint tube 3 serving as the tubular member as the insert member, and the gate portion 33, which is a portion of an inlet for pouring, or injecting, the molding material of the holder 15 into a molding die (see, e.g., FIGS. 7 to 10), is provided in the head portion 15b of the holder 15. Details of a step of pouring the molding material will be described later (see, e.g., FIG. 12).

When the gate portion 33 is provided at such a position, the molding material flows from the head portion 15b of the holder 15 toward the body portion 15a in the molding die. At that time, the molding material having a predetermined temperature or higher temperature (for example, 200° Celsius, or higher) is brought into contact with one end on the head portion 15b side of the tubular member as the insert member, for instance, the proximal end of the joint tube 3. Further, the molding material moves along the proximal end of the joint tube 3 and flows to the position where the outer circumferential contact portion 19 is formed and the position where the inner circumferential contact portion 20 is formed. In this manner, when the gate portion 33 is provided in the head portion 15b, the molding material having the predetermined temperature, or higher, easily comes into contact with the proximal end of the joint tube 3. Thus, the proximal end of the joint tube 3 is easily softened or melted by heat. A melting point of the joint tube 3 is preferably 120° Celsius, or lower. In this manner, it is possible to realize the joint tube 3 which is easily softened or melted by coming into contact with the molding material having the predetermined temperature, or higher temperature (for example, 200° Celsius, or higher). As an example of the joint tube 3 configured as above, it is possible to use the joint tube 3 having a melting point of about 95° Celsius and made of polybutadiene, for example. Further, the molding material having the predetermined temperature, or higher temperature, advances along the outer surface of the joint tube 3 from the proximal end of the joint tube 3 to the outer circumferential face of the joint tube 3 and flows into the position where the outer circumferential contact portion 19 is formed. Thus, the proximal end of the joint tube 3, which has been softened or melted by heat to be fluidized, is pushed by the flow of the molding material flowing to the position where the outer circumferential contact portion 19 is formed, thereby easily forming the protruding portion 25. As described above, since the gate portion 33 is provided in the head portion 15b, the convex portion serving as the protruding portion 25, which protrudes outward in the radial direction C, is easily formed at one end on the head portion 15b side of the tubular member, that is, the proximal end of the joint tube 3 at the time of integral molding. In addition, even after the convex portion serving as the protruding portion 25 is formed, the molding material has fluidity (e.g., in a softened flowing, or at least partially melted, state, etc.), and thus, swirls along an outer shape of the protruding portion 25. As a result, the outward annular convex portion serving as the protruding portion 25 is formed on the tubular member, and the outward annular concave portion serving as the recessed portion 26 and accommodating the outward annular convex portion is formed in the fixing member. That is, the inner wall, which defines the outward annular concave portion of the fixing member, is formed to serve as the movement restriction portion 11 that is engaged with the protruding portion 25 and causes the fixing member to be substantially prevented from being detached from the tubular member or vice versa.

The molding material having the predetermined temperature, or higher, and having flowed from the position of the gate portion 33 forms not only the flow advancing along the outer surface of the joint tube 3 from the proximal end of the joint tube 3 to the outer circumferential face of the joint tube 3 but also the flow advancing along the outer surface of the joint tube 3 from the proximal end of the joint tube 3 to the inner circumferential face of the joint tube 3. That is, there is also the flow that led into the position where the inner circumferential contact portion 20 is formed. Thus, the inward annular convex portion serving as the protruding portion 27 is formed on the tubular member, and the inward annular concave portion serving as the recessed portion 28 and accommodating the inward annular convex portion is formed in the fixing member according to the same principle as the protruding portion 25 and the recessed portion 26 described above. However, a volume of the projection 23 serving as the inner circumferential contact portion 20 in the present embodiment is smaller than a volume of the tube portion 21 serving as the outer circumferential contact portion 19, and the molding material flowing into the position where the inner circumferential contact portion 20 is formed is less than the molding material flowing into the position where the outer circumferential contact portion 19 is formed in its amount. Thus, in some embodiments, a protruding height H1 (see, e.g., FIG. 5) of the outward annular convex portion, which serves as the protruding portion 25, from the outer circumferential face of the body portion 3a of the joint tube 3 in the radial direction C is higher than a protruding height H2 (see, e.g., FIG. 5) of the inner annular convex portion, which serves as the protruding portion 27, from the inner circumferential face of the body portion 3a of the joint tube 3 in the radial direction C.

A volume of the head portion 15b is preferably larger than a volume of the body portion 15a. By setting the volume of the head portion 15b to be larger than the volume of the body portion 15a, it is possible to enhance the fluidity, or flowability, of the molding material at the head portion 15b and to enhance the internal pressure applied from the head portion 15b toward the body portion 15a when maintaining the pressure as compared with a case where the volume of the head portion is smaller than the volume of the body portion. As a result, it is possible to easily form the outward annular convex portion serving as the protruding portion 25, the outward annular concave portion serving as the recessed portion 26, the inward annular convex portion serving as the protruding portion 27, and the inward annular concave portion serving as the recessed portion 28 described above.

As illustrated in FIG. 3, the gate portion 33 remains as a small projection or the like on the outer surface of the integrally-molded holder 15 in some cases, and the position of the gate portion 33 can be identified from the integrally-molded medical device 1.

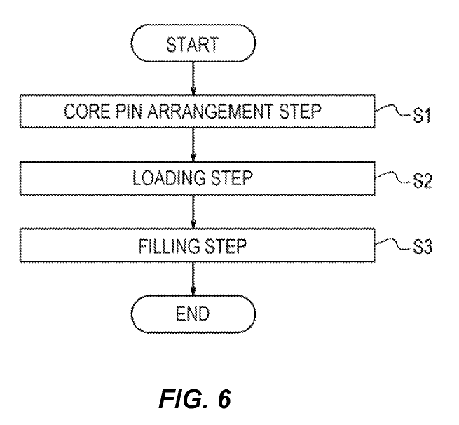

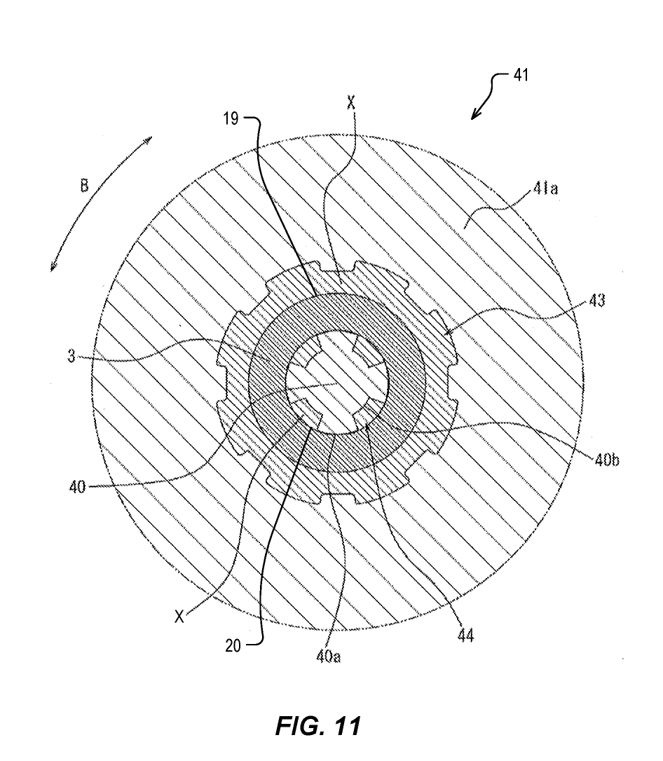

Finally, a method for manufacturing the medical device 1 will be described with reference to FIGS. 6 to 12 and in accordance with embodiments of the present disclosure. FIG. 6 is a flowchart illustrating a method for manufacturing the medical device 1. In addition, FIGS. 7 to 10 are schematic cross-sectional views illustrating a mold and an outline of each step of the method (e.g., the molding process, etc.) for manufacturing the medical device 1. Further, FIG. 11 is a schematic cross-sectional view taken along line I-I in FIG. 9. Furthermore, FIG. 12 is a schematic cross-sectional view illustrating a position of the inlet port for adding (e.g., injecting, etc.) the molding material when the medical device 1 is integrally molded according to embodiments of the present disclosure.

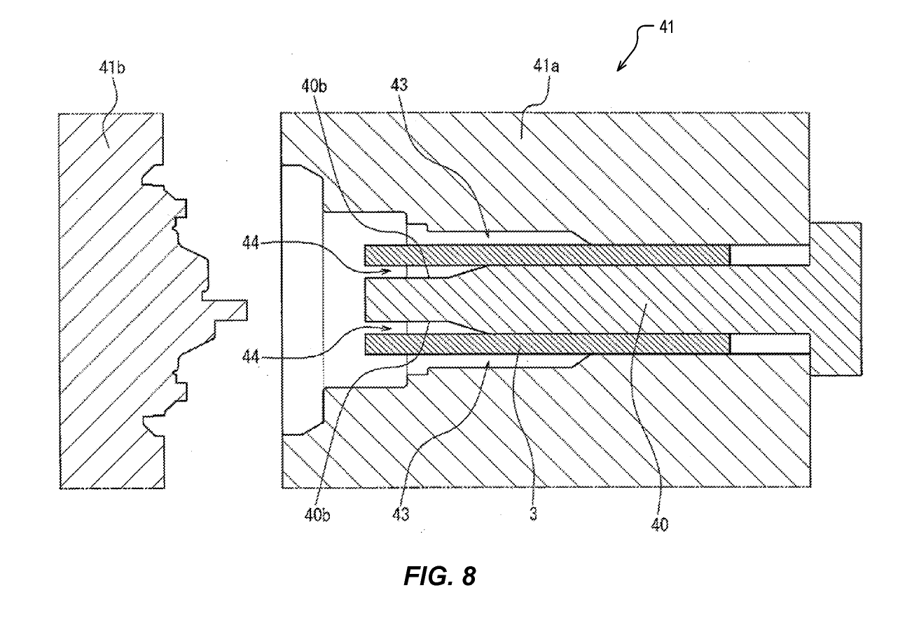

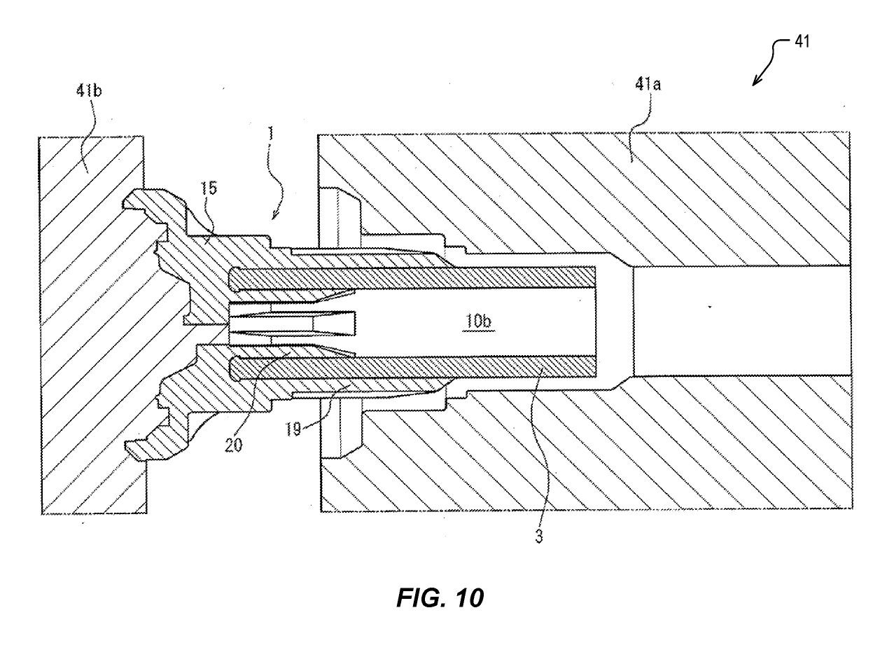

As illustrated in FIG. 6, the method for manufacturing the medical device 1 includes: a core pin arrangement step S1 of arranging a core pin to which a tubular member is externally fitted in a molding die forming an outer shape of a fixing member; a loading step S2 of loading the tubular member into the molding die; and a filling step S3 of filling the molding die with a molding material of the fixing member so as to be in contact with an inner circumferential face and an outer circumferential face of the tubular member and integrally molding the fixing member and the tubular member together. FIG. 7 illustrates an outline of the above-described core pin arrangement step S1. In addition, FIG. 8 illustrates an outline of the above-described loading step S2. Further, FIG. 9 illustrates an outline of the above-described filling step S3. Further, FIG. 10 illustrates a state where the medical device 1, in which the tubular member and the fixing member are integrally molded, is taken out of the molding die after solidification of the molding material that has been injected in the filling step S3. Hereinafter, the respective steps S1 to S3 of the method of manufacturing will be described.

As illustrated in FIG. 7, in the core pin arrangement step S1, the core pin 40 to which the tubular member is externally fitted is arranged in a molding die 41 forming the outer shape of the fixing member. As described above, the tubular member of the medical device 1 of the present embodiment may correspond to the joint tube 3.

As illustrated in FIG. 7, the molding die 41 includes: an annular die 41a that has an annular shape and forms an outer surface of the holder 15 serving as the fixing member (see, e.g., FIG. 3) in the radial direction C; and a lid-like die 41b that closes one end side of the annular die 41a and forms a face on the proximal end side of the holder 15. The core pin 40 is inserted into the inside of the molding die 41 from the other end side (e.g., opposite the lid-like die 41b side, etc.) of the annular die 41a. A cross-sectional shape of the core pin 40 will be described later in conjunction, for example, with FIG. 11, etc.

As illustrated in FIG. 8, in the loading step S2, the joint tube 3 as the insert member is externally fitted with respect to the core pin 40 arranged in the molding die 41 in the core pin arrangement step Sl. When the joint tube 3 is externally fitted, an outer flow path 43 is defined on the outer circumferential face side of the joint tube 3 in the molding die. In addition, an inner flow path 44 is defined on the inner circumferential face side of the joint tube 3. The outer flow path 43 and the inner flow path 44 may provide a space between the molding die and the joint tube 3 where the molding material may flow forming the holder 15.

After completion of the loading step S2 illustrated in FIG. 8, one end of the annular die 41a may be closed by the lid-like die 41b so that a die internal space S, or cavity, filled with a molding material X as illustrated in FIG. 9 is formed. When the lid-like die 41b is arranged after the loading step S2 as illustrated in FIG. 9, a distal end of the core pin 40 and the lid-like die 41b come into contact with one another. Further, a portion of the core pin 40 forms the flow path 10b (see, e.g., FIG. 10 and the like) of the integrally-molded holder 15.

As illustrated in FIG. 9, the die internal space S, or cavity, is filled with the molding material X in the filling step S3. The molding material X of the fixing member of the present embodiment may correspond to a thermoplastic resin or the like.

Then, the medical device 1 as the integrally-molded article is taken out of the die after the molding material X has been solidified, or cured, as illustrated in FIG. 10. Specifically, the core pin 40 is pulled out of the joint tube 3 serving as the tubular member. Next, the medical device 1 is taken out of the annular die 41a together with the lid-like die 41b. Thereafter, the lid-like die 41b is taken out of the medical device 1, thereby obtaining the medical device 1 as the integrally-molded article (e.g., comprising the holder 15 and the joint tube 3).

The cross-sectional shape of the core pin 40 will now be described. In a state where the joint tube 3 serving as the tubular member is externally fitted to the core pin 40, the core pin 40 includes a contact region 40a in contact with the inner circumferential face of the joint tube 3 and a flow path formation region 40b, which defines the inner flow path 44 against the inner circumferential face of the joint tube 3 without being in contact with the inner circumferential face of the joint tube 3, at different positions in the circumferential direction B as illustrated in FIG. 11.

More specifically, the core pin 40 of the present embodiment has a gear-like cross-sectional outer shape along a section of the core pin 40. In addition, a convex portion and a concave portion in the cross section of the core pin 40 of the present embodiment extend over the whole region in the longitudinal direction of the core pin 40. Thus, when the joint tube 3 is externally fitted to the core pin 40, the top of the convex portion of the core pin 40 comes into contact with the inner circumferential face of the joint tube 3 to hold the cross-sectional shape of the joint tube 3 in a substantially circular shape. On the other hand, a gap may be formed between the core pin 40 and the inner circumferential face of the joint tube 3 at a position of the concave portion. In some embodiments, the above-described contact region 40a is configured by the top of the convex portion of the core pin 40. Additionally or alternatively, the above-described flow path formation region 40b may be configured by the concave portion of the core pin 40. Further, the above-described inner flow path 44 is a gap defined by the concave portion of the core pin 40 and the joint tube 3.

As illustrated in FIGS. 9 and 11, the die internal space S, or cavity, defined by the core pin 40, the annular die 41a, and the lid-like die 41b is filled with the molding material X in the filling step S3. The molding material X that has been injected flows from a space forming the head portion 15b (see FIG. 3) of the holder 15 into a space forming the body portion 15a (see FIG. 3) of the holder 15. Specifically, the molding material X flows into the outer flow path 43 and the inner flow path 44 to form the tube portion 21 (see, e.g., FIG. 3) serving as the outer circumferential contact portion 19 and the projection 23 (see, e.g., FIG. 3) serving as the inner circumferential contact portion 20.

As illustrated in FIG. 11, a position in the circumferential direction B where the outer flow path 43 and the inner flow path 44 oppose each other in the radial direction becomes a position where the above-described pinching portion 31 (see, e.g., FIG. 3) is formed. On the other hand, a position in the circumferential direction B where the outer flow path 43 and the inner flow path 44 do not oppose each other in the radial direction becomes a position where the above-described non-pinching portion 32 (see, e.g., FIG. 3) is formed.

FIG. 12 is a view illustrating a position of an inlet 60 for injecting the molding material X in the filling step S3. That is, it is a view that illustrates a position of a portion, which is to serve as the gate portion 33 (see, e.g., FIG. 3). As illustrated in FIG. 12, the inlet 60 of the molding material X to serve as the gate portion 33 after integral molding is provided in the space where the head portion 15b (see, e.g., FIG. 3) of the holder 15 is formed. Thus, the protruding portion 25 (see, e.g., FIG. 5), the recessed portion 26 (see, e.g., FIG. 5), the protruding portion 27 (see, e.g., FIG. 5), and the recessed portion 28 (see, e.g., FIG. 5) are formed at the proximal end of the joint tube 3 by the flow of the molding material X having high temperature in the filling step S3 illustrated in FIG. 9, and it is possible to further strengthen the connection between the tubular member and the fixing member in the medical device 1 as the integrally-molded article.

The medical device and the method for manufacturing the medical device according to the present invention are not limited to the specific configurations described above, and various modifications can be made within a range not departing from the scope of the present disclosure. For example, the joint tube 3 connecting the first medical connector 2 and the medical tube 4 is illustrated as the tubular member of the medical device 1 in the above-described embodiment, but the medical tube 4 may be used as a tubular member to which a holder 15′ serving as a fixing member of a first medical connector 2′ is fixed without using the joint tube 3 as in a medical device 1′ illustrated in FIG. 13. In addition, each of the protruding portion 25, the recessed portion 26, the protruding portion 27, and the recessed portion 28 is formed in an annular shape in the above-described embodiment, but may have another shape, for example, a semicircular shape or the like.

The holder 15′ illustrated in FIG. 13 is different from the above-described holder 15 (see, e.g., FIG. 3) in terms of the configuration of the inner circumferential contact portion 20. The above-described holder 15 may be configured such that the outer circumferential contact portion 19 is longer toward the distal end side of the joint tube 3 serving as the tubular member than the inner circumferential contact portion 20, whereas the holder 15′ illustrated in FIG. 13 may be configured such that positions of the distal ends of the outer circumferential contact portion 19 and the inner circumferential contact portion 20 in the central axis direction A are substantially equal. More specifically, the position of the distal end of the tube portion 21 serving as the outer circumferential contact portion 19 and the position of the distal end of a projection 23′ serving as the inner circumferential contact portion 20 are substantially equal in the central axis direction A in FIG. 13. In this manner, it is also possible to make the positions in the central axis direction A of the distal ends of the outer circumferential contact portion 19 and the inner circumferential contact portion 20 substantially equal (e.g., lying along a same plane in the radial direction C, etc.). However, when the joint tube 3 connecting the fixing member and the medical tube 4 is used as the tubular member as described above, it may be beneficial to have the configuration in which the outer circumferential contact portion 19 extends longer so as to protrude from the inner circumferential contact portion 20 and the medical tube 4 is accommodated inside the joint tube 3 and inside the holder 15. In this manner, it is possible to prevent the connection between the joint tube 3 and the medical tube 4 from being loosened or detached as described above.

In addition, the method for manufacturing the medical device illustrated in FIG. 6 includes the core pin arrangement step S1 of arranging the core pin to which the tubular member is externally fitted in the molding die forming the outer shape of the fixing member, but a core pin may be formed integrally with a molding die and the core pin arrangement step S1 may be omitted, for example, without being limited to the above method. Even in such a case, it is possible to externally fit a tubular member to the core pin to form an inner flow path between an inner circumferential face of the tubular member and the core pin, and to form an outer flow path on an outer circumferential face side of the tubular member in the loading step S2. Then, it is possible to fill the inner flow path and the outer flow path with the molding material in the filling step S3 and to manufacture a medical device.

The present disclosure relates to a medical device and a method for manufacturing the medical device.

### DESCRIPTION OF REFERENCE CHARACTERS

1, 1′ medical device

2, 2′ first medical connector

3 joint tube (tubular member)

3a body portion

4 medical tube

5 second medical connector

6 indwelling needle member

7 clamp

10 hollow portion

10a cap hollow portion

10b flow path

11 movement restriction portion

12 housing

13 elastic valve body

13a top face

13b bottom face

14 cap

14a tube portion

14b flange portion

15, 15′ holder (fixing member)

16 top face cap

17 bottom face cap

18 slit

19 outer circumferential contact portion

20 Inner circumferential contact portion

21 tube portion

22 annular flange portion

23, 23′ projection

23a tubular proximal end

23b protruding distal end

24 annular groove portion

25 protruding portion

26 recessed portion

27 protruding portion

28 recessed portion

29 hub member

30 indwelling needle

31 pinching portion

32 non-pinching portion

33 gate portion

40 core pin

40a contact region

40b flow path formation region

41 molding die

41a annular die

41b lid-like die

43 outer flow path

44 inner flow path

50 tubular portion

60 inflow port

100 infusion set

A central axis direction of tubular member

B circumferential direction of tubular member

C radial direction of tubular member

O central axis

T1 thickness of outer circumferential contact portion in the radial direction

T2 thickness of inner circumferential contact portion in the radial direction

H1, H2 protruding height of protruding portion in radial the direction

S die internal space

X molding material

What is claimed is:

1. An integrally-molded medical device, comprising:
a tubular member; and
a fixing member molded to the tubular member and fixed to a first end of the tubular member,
wherein the fixing member comprises an outer circumferential contact portion in contact with an outer circumferential face of the tubular member, and an inner circumferential contact portion in contact with an inner circumferential face of the tubular member, and
a circumferential wall of the tubular member comprises, at different positions in a circumferential direction, a pinching portion which is pinched by and between the outer circumferential contact portion and the inner circumferential contact portion, and a non-pinching portion which is in contact with the outer circumferential contact portion, is not in contact with the inner circumferential contact portion, and is not pinched by the outer circumferential contact portion and the inner circumferential contact portion.

2. The medical device according to claim 1, wherein at a position where the pinching portion is formed, a thickness of the outer circumferential contact portion in a radial direction of the tubular member is thicker than a thickness of the inner circumferential contact portion in the radial direction.

3. The medical device according to claim 2, wherein a minimum inner diameter of the outer circumferential contact portion is smaller than an outer diameter of the tubular member in a natural state of the tubular member.

4. The medical device according to claim 2, wherein the outer circumferential contact portion covers the outer circumferential face of the tubular member in a whole region in the circumferential direction.

5. The medical device according to claim 1, wherein the outer circumferential contact portion is longer toward a second end of the tubular member than the inner circumferential contact portion, wherein the second end of the tubular member is disposed opposite the first end of the tubular member.

6. The medical device according to claim 1, wherein the fixing member comprises:
a body portion molded at a position overlapping the first end of the tubular member in a central axis direction of the tubular member;
a head portion extending in the central axis direction from the body portion and molded at a position that is not overlapping the tubular member; and
a gate portion disposed in the head portion of the fixing member, wherein the gate portion is used during integral molding of the fixing member to the tubular member.

7. The medical device according to claim 6, wherein a volume of the head portion is larger than a volume of the body portion.

8. An integrally-molded medical device, comprising:
a tubular member; and
a fixing member fixed to a first end of the tubular member,
wherein the fixing member comprises an outer circumferential contact portion in contact with an outer circumferential face of the tubular member, and an inner circumferential contact portion in contact with an inner circumferential face of the tubular member,
a circumferential wall of the tubular member comprises a pinching portion which is pinched by the outer circumferential contact portion and the inner circumferential contact portion, and
at a position where the pinching portion is formed, a thickness of the outer circumferential contact portion in a radial direction of the tubular member is thicker than a thickness of the inner circumferential contact portion in the radial direction.

9. The medical device according to claim 8, wherein the fixing member comprises:
a body portion provided at a position overlapping the first end of the tubular member in a central axis direction of the tubular member;
a head portion extending in the central axis direction from the body portion and provided at a position that is not overlapping the tubular member; and
a gate portion disposed in the head portion of the fixing member, wherein the gate portion is used during integral molding of the fixing member to the tubular member.

10. The medical device according to claim 8, wherein the first end of the tubular member comprises a protruding portion that protrudes radially outward from the central axis, and wherein the fixing member comprises a movement restriction portion which is in contact with a face of the protruding portion of the tubular member, and restricts movement of the first end of the tubular member in a removal direction, wherein the removal direction is in a direction running along the central axis from the first end of the tubular member toward a second end of the tubular member disposed opposite the first end of the tubular member.

11. The medical device according to claim 8, wherein a minimum inner diameter of the outer circumferential contact portion is smaller than an outer diameter of the tubular member in a natural state of the tubular member.

12. The medical device according to claim 8, wherein the circumferential wall of the tubular member comprises, at different points in a circumferential direction, a series of pinching portions which are pinched by and between the outer circumferential contact portion and the inner circumferential contact portion, and a series of non-pinching portions which are in contact with the outer circumferential contact portion but not in contact with the inner circumferential contact portion.

13. The medical device according to claim 9, wherein a volume of the head portion is larger than a volume of the body portion.

14. A method for manufacturing an integrally-molded medical device which includes a tubular member and a fixing member fixed to a first end of the tubular member, the method comprising:
a loading step of loading the tubular member into a molding die including a cavity that forms an outer shape of the fixing member;
a filling step of filling the molding die with a molding material of the fixing member so as to be in contact with an inner circumferential face and an outer circumferential face of the tubular member and integrally molding the fixing member and the tubular member together, wherein, after the molding material of the fixing member cures, the inner circumferential face and the outer circumferential face of the tubular member are pinched between portions of the fixing member at the first end of the tubular member.

15. The method for manufacturing the medical device according to claim 14, wherein, in the loading step, the tubular member is externally fitted to a core pin, and an inner flow path is formed between the inner circumferential face of the tubular member and the core pin, and an outer flow path is formed on a side of the outer circumferential face of the tubular member, and wherein, in the filling step, the inner flow path and the outer flow path are filled with the molding material.

16. The method for manufacturing the medical device according to claim 14, wherein the fixing member comprises:
a body portion provided at a position overlapping the first end of the tubular member in a central axis direction of the tubular member;
a head portion extending in the central axis direction from the body portion and provided at a position that is not overlapping the tubular member; and
a gate portion disposed in the head portion of the fixing member, wherein, in the filling step, the gate portion is used to receive the molding material of the fixing member in a fluid state.

17. The method for manufacturing the medical device according to claim 16, wherein the first end of the tubular member comprises a protruding portion that protrudes radially outward from the central axis and has a thickness in a direction running along the central axis, and wherein, in the filling step, the molding material is injected at the gate portion and caused to swirl inside the cavity and along an outer shape of the protruding portion forming a movement restriction portion of the fixing member in contact with a face of the protruding portion of the tubular member, and wherein, after the molding material of the fixing member cures, the movement restriction portion of the fixing member in contact with the face of the protruding portion of the tubular member restricts movement of the tubular member relative to the fixing member in a removal direction toward a second end of the tubular member disposed opposite the first end of the tubular member.

18. The medical device according to claim 1, wherein at a position where the pinching portion is formed, a thickness of the outer circumferential contact portion of the fixing member in a radial direction of the tubular member is thicker than a thickness of the inner circumferential contact portion of the fixing member in the radial direction.

19. The medical device according to claim 1, wherein the first end of the tubular member comprises a protruding portion that protrudes radially outward from the central axis, and wherein the fixing member comprises a movement restriction portion that is in contact with a face of the protruding portion of the tubular member.

20. The medical device according to claim 19, wherein the movement restriction portion in contact with the face of the protruding portion of the tubular member restricts movement of the first end of the tubular member in a removal direction along the central axis from the first end of the tubular member toward a second end of the tubular member disposed opposite the first end of the tubular member.
